(19) United States
(12) Patent Application Publication
(10) Pub. No.: US 2012/0202945 A1
(43) Pub. Date: Aug. 9, 2012

(54) POLYURETHANES AND USE THEREOF

(75) Inventors: Manfred Schmidt, Dormagen (DE); Robert Vieler, Dormagen (DE); Ernst Felske, Neuss (DE); Hans-Georg Pirkl, Leverkusen (DE); Jens Krause, Leverkusen (DE); Arnaud Vilbert, Bourg de Peage (FR)

(73) Assignees: Baulé S.A.S., Romans Sur Isère (FR); Bayer MaterialScience AG, Leverkusen (DE)

(21) Appl. No.: 13/382,817

(22) PCT Filed: Jun. 25, 2010
(86) PCT No.: PCT/EP2010/003910

§ 371 Date: Apr. 19, 2012

(30) Foreign Application Priority Data
Jul. 7, 2009 (FR) 0954679

(51) Int. Cl. C09D 175/04; C08G 18/24; C07C 265/14; C08G 18/28

(52) U.S. Cl. 524/590; 528/76; 528/48; 528/58; 560/359

(57) ABSTRACT

The invention relates to polyurethanes that can be obtained by reacting a prepolymer based on diphenylmethane diisocyanate (MDI) with compounds that have NCO-reactive groups, and to use thereof.

The invention relates to polyurethanes which are obtainable by reaction of a diphenylmethane diisocyanate (MDI)-based prepolymer with compounds containing NCO reactive groups, and also to the use thereof.

In the insulation of pipes, particularly in the case of deep-sea pipes, syntactic polyurethanes are employed. The term “syntactic polymers” generally encompasses plastics which comprise hollow fillers. Syntactic polymers typically find use, on account of their advantageous compressive strength and temperature stability, as thermal insulation coatings, preferably in the offshore segment. Other applications in the offshore segment are bend stiffeners, bend restrictors, buoys, clamp systems, cables, flow traversal systems and ballast tanks. Besides the offshore applications, polyurethanes of this kind are also employed in the spraying area, on building sites, for example, such as for the production of concrete formwork mats. Another typical outdoor application is that of reactive polyurethane adhesives as well. Likewise known are applications as fire protection material and as sound insulation material.

In the aforementioned applications it is preferred to use diphenylmethane diisocyanate (MDI) based systems. MDI consists primarily of the isomers 2,4′-MDI, 4,4′-MDI, and 2,2′-MDI. Often times desired is a maximally high fraction of pure 4,4′-MDI, since 4,4′-MDI gives the best mechanical properties for the prepolymers/polyurethanes and, furthermore, is the most inexpensive. Disadvantages of 4,4′-MDI include its high crystallization tendency and its high melting point. By modifying the 4,4′-MDI, such as by carbodiimidization, urethanization (essentially prepolymerization), and allophanatization, for instance, the crystallization tendency of the 4,4′-MDI can be reduced. The objective here is to obtain a product with a very high NCO content (i.e., slight modification), which possesses a low viscosity and at the same time is liquid throughout the outdoor temperature range. This outdoor temperature range is −15° C. to 50° C.

A modification of he MDI is in principle a reaction of the NCO group of the MDI. The formation of a prepolymer is a special case of a modification, and relates to the reaction of a compound containing NCO reactive groups with the NCO groups of the MDI.

Disadvantages of numerous modifications, however, are that
a) carbodiimidization of the MDI greatly increases the functionality; often, however, linear systems with a functionality of 2 are required;
b) modification often lowers the NCO content so sharply that the viscosity at room temperature becomes too high; however, products which are liquid at room temperature, with a viscosity of <2000 mPas at 25° C., are advantageous; this usually corresponds to an NCO content of >22%;
c) the modification does indeed allow the product to be processed in a broad temperature range, but the product tends toward crystallization of the remaining free MDI in the MDI prepolymers; in order to avoid this, it is common to add polycyclic MDI (also known as polymeric MDI) to the prepolymer; as a result of this, however, the functionality is increased and the mechanical properties are impaired, and so, in turn, greater amounts of polymeric MDI are needed in order to improve the crystallization stability; greater amounts of polymeric MDI, however, increase not only the functionality but also—greatly—the viscosity.

A further disadvantage of numerous modifications, moreover, is that they are obtained by reaction with expensive tripropylene glycol.

An object of the invention, therefore, was to provide a modified MDI based prepolymer which possesses a viscosity at 25° C. of <2000 mPas, is liquid down to −10° C., and does not crystallize after 2 months of storage at −5° C., being obtained not by reaction with tripropylene glycol, but instead by means of more inexpensive polyols.

The invention provides polyurethanes obtainable from
a) a prepolymer containing isocyanate groups and based on diphenylmethane diisocyanate, with an NCO content of 23% to 28% by weight,
obtained from
(i) at least one polyol based on propylene oxide, with an OH number of 200 to 1000, preferably 400 to 800, more preferably 500 to 700 mg KOH/g and a functionality of 1.8 to 4, with the exception of tripropylene glycol, and optionally containing 1,3-butanediol and/or 1,2-propylene glycol in amounts each of max. 2% by weight, based on (a), and
(ii) a mixture of 4,4′-diphenylmethane diisocyanate (4,4′-MDI) modified with carbodiimide groups and uretonimine groups, the uretonimine groups content being 10% to 40% by weight, based on (ii), and a mixture of 15% to 40% by weight of 2,4′-MDI, 60% to 85% by weight of 4,4′-MDI and 0% to 10% by weight of 2,2′-MDI,
b) at least one compound containing NCO-reactive groups, preferably polyether polyols, more preferably polyoxypropylene polyols, having a functionality of 1.8 to 3 and an OH number of 20 to 150,
c) at least one chain extender and/or crosslinking agent having a functionality of 2 to 3 and a molecular weight of 62 to 500
in the presence of
d) catalysts,
e) optionally auxiliaries and/or additives,
f) optionally epoxy resins in amounts from 2% to 10% by weight, based on polyurethane.

Surprisingly it has been found that by reacting polyols based on propylene oxide with 2,4′-MDI enriched MDI in the presence of carbodiimide/uretonimine-modified 4,4′-MDI, it is possible to provide crystallization-stable NCO prepolymers of low viscosity. Particularly preferred are NCO prepolymers obtained by reaction of carbodiimide/uretonimine-modified 4,4′-MDI and 2,4′-MDI enriched MDI (mixed with 2,4′/4,4′-MDI) in one step with the polyol based on propylene oxide. The continuous preparation of the prepolymer is particularly preferred.

The MDI based prepolymers are notable for their low viscosity at room temperature and for their particular low-temperature stability. These prepolymers, therefore, and/or the polyurethanes prepared from them, are used preferably also in outdoor applications directly on site, such as on building sites in the case of spraying applications, for example. The polyurethanes are also used for producing products in the offshore segment, examples being offshore pipes, and other components and devices used in the offshore segment, that are insulated with the polyurethane of the invention.

The NCO prepolymer used in accordance with the invention begins to crystallize only at below 0° C., preferably below −5° C., after 2 months of storage in closed containers. The viscosity of this NCO prepolymer at 25° C. in accordance with DIN EN ISO 11909 is less than 2000 meas.

The invention further provides prepolymers containing isocyanate groups and based on diphenylmethane diisocyanate, having an NCO content of 23% to 28% by weight, obtainable from
(i) at least one polyol based on propylene oxide, with an OH number of 200 to 1000, preferably 400 to 800, more preferably 500 to 700 mg KOH/g and a functionality of 1.8 to 4, with the exception of tripropylene glycol, and optionally containing 1,3-butanediol and/or 1,2-propylene glycol in amounts each of max. 2% by weight, based on (a), and
(ii) a mixture of 4,4′-diphenyhnethane diisocyanate (4,4′-MDI) modified with carbodiimide groups and uretonimine groups, the uretonimine groups content being 10% to 40% by weight, based on (ii), and a mixture of 15% to 40% by weight of 2,4′-MDI, 60% to 85% by weight of 4,4′-MDI and 0% to 10% by weight of 2,2′-MDI.

With preference, hollow microspheres can be used in the polyurethane as an additive when syntactic polyurethanes are to be prepared.

The term “hollow microsphere” in the context of this invention refers to organic and mineral hollow spheres. Organic hollow spheres that may be used include, for example, hollow plastics spheres, made of polyethylene, polypropylene, polyurethane, polystyrene or a mixture thereof, for example. Hollow mineral spheres may be produced, for example, on the basis of clay, aluminum silicate, glass or mixtures thereof. The hollow spheres may have a vacuum or partial vacuum in the interior or may be filled with air, inert gases, such as nitrogen, helium or argon, for example, or reactive gases, such as oxygen, for example. The organic or mineral hollow spheres preferably have a diameter of 1 to 1000 μm, preferably of 5 to 200 μm. The organic or mineral hollow spheres preferably have a bulk density of 0.1 to 0.4 g/cm3. They generally possess a thermal conductivity of 0.03 to 0.12 W/mK. As hollow microspheres it is preferred to use hollow glass microspheres. In one particularly preferred embodiment, the hollow glass microspheres have a hydrostatic compressive strength of at least 20 bar. As hollow glass microspheres it is possible, for example, to use 3M Scotchlite® Glass Bubbles. As plastics-based hollow microspheres it is possible, for example, to use Expancel products from Akzo Nobel.

Chain extenders/crosslinking agents used are compounds having a functionality of 2 to 3 and a molecular weight of 62 to 500. Use may be made of aromatic aminic chain extenders such as, for example, diethyltoluenediamine (DETDA), 3,3′-dichloro-4,4′-diaminodiphenylmethane (MBOCA), 3,5-diamino-4-chloroisobutyl benzoate, 4-methyl-2,6-bis(methylthio)-1,3-diaminobenzene (Ethacure 300), trimethylene glycol di-p-amino benzoate (Polacure 740M), and 4,4′-diamino-2,2′-dichloro-5,5′-diethyldiphenylmethane (MCDEA). Particularly preferred are MBOCA and 3,5-diamino-4-chloroisobutyl benzoate. Aliphatic aminic chain extenders may likewise be employed or used as well. These often have a thioxotropic effect on the basis of their high reactivity. Examples of nonaminic chain extenders often used include 2,2′-thiodiethanol, propane-1,2-diol, propane-1,3-diol, glycerol, butane-2,3-diol, butane-1,3-diol, butane-1,4-diol, 2-methylpropane- 1,3-diol, pentanediol-1,2, pentane-1,3 -diol pentane-1,4,diol pentanediol-1,5,2,2-dimethylpropane-1,3diol, 2-methylbutane-1,4-diol, 2-methylbutane-1,3-diol, 1,1,1-trimethylol-ethane, 3-methyl-1,5-pentanediol, 1,1,1-trimethylolpropane, 1,6-hexanediol, 1,7-heptanediol, 2-ethyl-1,6-hexanediol, 1,8-octanediol, 1,9-nonanediol, 1,10-decanediol 1,11-undecanediol, 1,12-dodecanediol, diethylene glycol, triethylene glycol, 1,4-cyclohexanediol, 1,3-cyclohexanediol, and water.

In preparing polyisocyanate polyaddition products it is possible with preference, as NCO-reactive compounds, to use polyols having OH numbers in a range from 20 to 150, preferably 27 to 150, more preferably 27 to 120 mg KOH/g and an average functionality of 1.8 to 3, preferably 1.8 to 2.4. Polyols used may be polyether polyols, polyester polyols, polycarbonate polyols, and polyetherester polyols.

Polyether polyols are prepared either by means of alkaline catalysis or by means of double metal cyanide catalysis or optionally, in the case of a stagewise reaction regime, by means of alkaline catalysis and double metal cyanide catalysis, from a starter molecule and epoxides, preferably ethylene oxide and/or propylene oxide, and have terminal hydroxyl groups. Starters suitable in this context include the compounds with hydroxyl groups and/or amino groups that are known to the skilled person, and also water. The functionality of the starters in this case is at least 2 and at most 4. It is of course also possible to use mixtures of two or more starters. Mixtures of two or more polyether polyols can also be employed as polyether polyols.

Polyester polyols are prepared in a conventional way by polycondensation from aliphatic and/or aromatic polycarboxylic acids having 4 to 16 carbon atoms, optionally from their anhydrides, and optionally from their low molecular mass esters, including cyclic esters, with polyols predominantly of low molecular weight and having 2 to 12 carbon atoms being employed as a reaction component. The functionality of the synthesis components for polyester polyols in these cases is preferably 2, but in certain cases may also be greater than two, the components with functionalities of more than 2 being used only in small amounts, so that the arithmetic number-average functionality of the polyester polyols in the range from 2 to 2.5, are obtained in accordance with the prior art from carbonic acid derivatives, as for example dimethyl carbonate or diphenyl carbonate or phosgene, and polyols by means of polycondensation.

Optionally it is possible to add adjuvants as well to the polyurethane, preferably by way of the compound containing NCO reactive groups. Mention may be made here, for example, of catalysts (compounds which accelerate the reaction of the isocyanate component with the polyol component), surface-active substances, dyes, pigments, hydrolysis protection stabilizers and/or antioxidants, and also UV protectants and epoxy resins. It is possible, furthermore, to add the blowing agents known from the prior art. It is preferred, however, for the isocyanate component and the compound containing the NCO reactive groups not to contain any physical blowing agent. It is also preferred that no water is added to the components a) and b). With particular preference, therefore, the components contain no blowing agent, apart from minimal amounts of residual water that is present in polyols produced industrially. It is also possible for the residual water content to be reduced by addition of water scavengers. Zeolites are examples of suitable water scavengers. The water scavengers are used, for example, in an amount of 0.1 to 10% by weight, based on the total weight of the compound containing the NCO reactive groups. The components a) and b) are mixed typically at a temperature of 0° C. to 100° C., preferably 15 to 60° C., and reacted. Mixing may take place with the customary PU processing machines. In one preferred embodiment, mixing is accomplished by low-pressure machines or high-pressure machines.

Catalysts known as latent catalysts (mercury compounds) are employed with preference. One known representative is phenylmercury neodecanoate (Thorcat 535 and Cocure 44). This catalyst displays a latent reaction profile, with the catalyst being virtually inactive to start with and becoming active suddenly only after slow heating of the mixture, usually by virtue of the exothermic heat of the uncatalyzed reaction of NCO with OH groups, at a particular temperature (usually around 70° C.). When such catalysts are used, it is possible to achieve very long open times with very short cure times. This is especially advantageous when a very large quantity of material has to be delivered (for example, when a large mold has to be filled), and when, after delivery has taken place, the reaction is to be ended quickly and hence economically.

When using latent catalysts it is particularly advantageous if, furthermore, the following conditions are met:
a) An increase in the quantity of catalyst accelerates the reaction, without the catalyst losing latency.
b) A reduction in the quantity of catalyst slows down the reaction, without the catalyst losing latency.
c) A variation in the quantity of catalyst, in the index, in the mixing ratio, in the discharge quantity and/or in the hard-segment fraction in the polyurethane does not impair the latency of the catalyst.
d) With all of the aforesaid variations, the catalyst ensures virtually complete conversion of the reactants, without sticky areas being left.

One particular advantage of the latent catalysts can be seen in the fact that in the completed polyurethane material, as a consequence of their decreasing catalytic effect with falling temperature, they have little accelerating effect on the cleavage of urethane groups, for example, at room temperature in comparison to conventional catalysts. They therefore contribute to favorable long-term service properties on the part of the polyurethanes. A review of the prior art is given in WO 2005/058996. There it is described how titanium catalysts and zirconium catalysts are employed. In addition, numerous possible combinations of different catalysts are mentioned.

Systems which are less toxic than mercury catalysts, based for example on tin, zinc, bismuth, titanium or zirconium, or amidine catalysts and amine catalysts, are indeed known in the market, but to date do not have the robustness and simplicity of the mercury compounds.

The effect of certain combinations of catalysts is that the gel reaction takes place very separately from the curing reaction, since many of these catalysts only act selectively. For example, bismuth(III) neodecanoate is combined with zinc neodecanoate and neodecanoic acid. Often, in addition, 1,8-diazabicyclo[5.4.0]undec-7-ene is added as well. Although this combination is among the best-known, it is, unfortunately, not so broadly and universally employable as, for example, Thorcat 535 (from Thor Especialidades S.A.), and, moreover, is sensitive to fluctuations in formulation. The use of these catalysts is described in DE 10 2004 011 348. Further combinations of catalysts are disclosed in WO 2005/058996, U.S. Pat. No. 3,714,077, U.S. Pat. No. 4,584,362, U.S. Pat. No. 5,011,902, U.S. Pat. No. 5,902,835, and U.S. Pat. No. 6,590,057.

It is preferred to use tin compounds such as DBTL (dibutyltin dilaurate), but very preferably tetravalent monocyclic tin compounds of the formula I having at least one ligand attached via at least one oxygen or sulfur atom and containing at least one nitrogen, i.e.

Sn(IV)(L¹)ₙ₁(L²)ₙ₂(L³)ₙ₃(L⁴)ₙ₄ (I)
with n1, n2, n3, and n4 being 0 or 1 and L¹, L², L³, and L⁴ being mono-, bi-, tri- or tetradentate ligands
or tetravalent polycyclic tin compounds based thereon, with at least one ligand per Sn atom having the following definition:

—X—Y
where X═O, S, OC(O), OC(S), O(O)S(O)O, O(O)S(O)

Y═—R1—N(R2)(R3) or —R1—C(R4)=NR2
R1, R2, R3, and R4 independently of one another being saturated or unsaturated, cyclic or acyclic, branched or unbranched, substituted or unsubstituted hydrocarbon radicals optionally interrupted by heteroatoms, or R2, R3, and R4 independently of one another being hydrogen or R1—X, or R2 and R3 or R2 and RI or R3 and R1 or R4 and R1 or R4 and R2 forming a ring,
and with the remaining ligands independently of one another being —X—Y with the aforesaid definition, or having the following definition:
   saturated or unsaturated, cyclic or acyclic, branched or unbranched, substituted or unsubstituted hydrocarbon radicals optionally interrupted by heteroatoms, or else halides, hydroxide, amide radicals, oxygen, sulfur, R2 or XR2, more preferably oxygen, sulfur, alcoholates, thiolates or carboxylates.

The invention further provides for the use of the syntactic polyurethanes of the invention for insulating offshore pipes or for producing sleeves for offshore pipes, and also for producing or coating other components and devices in the offshore segment.

Examples of other components and devices in the offshore segment are generators, pumps, and buoys. By offshore pipe is meant a pipe which is used for conveying oil and gas. In this context, the oil/gas is conveyed from the sea floor to platforms, into boats/tankers, or else directly into land. Sleeves are the connections between two pipes or pipe components.

The invention will be illustrated in more detail by the examples which follow.

### EXAMPLES

Starting Compounds:
Isocyanate 1 (Iso 1): 4,4′-diphenylmethane diisocyanate (4,4′-MDI), Desmodur® 44M from Bayer MaterialScience AG
Isocyanate 2 (Iso 2): uretonimine containing 4,4′-MDI, Desmodur® CD-S from Bayer MaterialScience AG
Isocyanate 3 (Iso 3): mixture of about 2% by weight 2,2′-diphenylmethane diisocyanate (2,2′-MDI), about 53% by weight 2,4′-diphenylmethane diisocyanate (2,4′-MDI), and about 47% by weight 4,4′-diphenylmethane diisocyanate (4,4′-MDI)
Isocyanate 4 (Iso 4): isocyanate mixture consisting of about 90% by weight 2,4′/4,4′-MDI and higher homologues of the diphenylmethane series
Polyether 1: polyether prepared from 1,2-propylene glycol and propylene oxide, having an OH number of 515 mg KOH/g
Polyether 2: tripropylene glycol

### Example 1

### Inventive

Preparation of Prepolymer 1 (Prep1):

56.1% by weight Iso 2

36.3% by weight Iso 3

7.6% by weight polyether 1

The isocyanates were introduced to start with. Thereafter the polyether was added. Stirring took place at 80° C. for 2 hours. The NCO content is 25.5% by weight and the viscosity at 25° C. is 170 mPa*s.

### Example 2

### Comparative

Preparation of Prepolymers 2 (Prep2):

18.4% by weight Iso 1

50% by weight Iso 2

26.2% by weight Iso 3

3.5% by weight polyether 2

The isocyanates were introduced to start with. Thereafter the polyether and also 1.1% by weight 1,3-butanediol and 0.8% by weight 1,2-propylene glycol were added. Stirring took place at 80° C. for 2 hours. The NCO content is 26% by weight and the viscosity at 25° C. is 200 mPa*s.

### Example 3

### Comparative

Preparation of Prepolymer 3 (Prep 3):

44% by weight Iso 1

50% by weight Iso 2

6.6% by weight polyether 2

Isocyanate 1 was introduced to start with. Thereafter the polyether was added. Stirring took place at 80° C. for 2 hours. Then blending took place with Iso 2. The NCO content is 26% by weight and the viscosity at 25° C. is 130 mPa*s.

### Example 4

### Comparative

Preparation of Prepolymer 4 (Prep 4):

89% by weight Iso 4

11% by weight polyether 1

The isocyanate was introduced to start with. Thereafter the polyether was added. Stirring took place at 80° C. for 2 hours. The NCO content is 24.5% by weight and the viscosity at 25° C. is 450 mPa*s.

### Example 5

### Comparative

Preparation of Prepolymer 5 (Prep 5):

92.5% by weight Iso 2

7.5% by weight polyether 1

The isocyanate was introduced to start with. Thereafter the polyether was added. Stirring took place at 80° C. for 2 hours. The NCO content is 24.5% by weight and the viscosity at 25° C. is 230 mPa*s.

The prepolymers crystallized at the following temperatures after two months of storage:

Prep 1 at −20° C.

Prep 2 at 5° C.

Prep 3 at 15° C.

Prep 4 at −5° C.

Prep 5 at −5° C.

### Application Examples

In each case 100 parts by weight of prepolymer were reacted with the amount of polyol given in the table (OH number 27 mg KOH/g solids; functionality 3) and also with 1,4-butanediol. The catalyst used was DBTL (dibutyltin dilaurate). The amounts of polyol and of 1,4-butanediol were varied such that all of the polyurethanes had the same hardness.

In comparison to the comparative polyurethanes, the inventive polyurethane elastomer exhibits an improved modulus at 10% and 100% elongation. The elongation, at 215%, is likewise high, and the tear propagation resistance, at 58 kN/m, is at a likewise very high level.

TABLE

| | | | Example 1 | Example 2 | Example 4 |
|---|---|---|---|---|---|
| Prepolymer formed from: | | | (invention) | (comparison) | (comparison) |
| 1,4-Butanediol [parts by | | | 24.5 | 24.3 | 22.9 |
| weight] | | | | | |
| Polyol [parts by weight] | | | 90 | 100 | 94.2 |
| DBTL [parts by weight] | | | 0.15 | 0.75 | 0.25 |
| Potlife | | [min] | 3′30 | 3′30 | 3′30 |
| Demolding time | | [min] | 20 | 15 | 20 |
| Hardness | DIN | Shore | 58D | 58D | 58D |
| | 53505 | | | | |
| 10% modulus | DIN | [MPa] | 12.6 | 10.8 | 11.6 |
| | 53504 | | | | |
| 100% modulus | DIN | [MPa] | 18.6 | 18.1 | 17.9 |
| | 53504 | | | | |
| 200% modulus | DIN | [MPa] | 21.1 | — | 22.8 |
| | 53504 | | | | |
| Strain at break | DIN | [MPa] | 22 | 20 | 24 |
| | 53504 | | | | |
| Elongation at break | DIN | [%] | 215 | 180 | 218 |
| | 53504 | | | | |
| Tear propagation resistance | DIN | [kN/m] | 58 | 51 | 57 |
| | 53515 | | | | |
| Rebound elasticity | DIN | [%] | 44 | 45 | 45 |
| | 53512 | | | | |
| Abrasion | DIN | [mm³] | 90 | 95 | 70 |
| | 53516 | | | | |

1-6. (canceled)

7. A polyurethane obtainable from
a) a prepolymer containing isocyanate groups and based on diphenylmethane diisocyanate, with an NCO content of 23% to 28% by weight, obtainable from
(i) at least one polyol based on propylene oxide, with an OH number of 200 to 1000, preferably 400 to 800, more preferably 500 to 700 mg KOH/g and a functionality of 1.8 to 4, with the exception of tripropylene glycol, and optionally containing 1,3-butanediol and/or 1,2-propylene glycol in amounts each of max. 2% by weight, based on (a), and
(ii) a mixture of 4,4′-diphenylmethane diisocyanate (4,4′-MDI) modified with carbodiimide groups and uretonimine groups, the uretonimine groups content being 10% to 40% by weight, based on (ii), and a mixture of 15% to 40% by weight of 2,4′-MDI, 60% to 85% by weight of 4,4′-MDI and 0% to 10% by weight of 2,2′-MDI,
b) at least one compound containing NCO-reactive groups, preferably polyether polyols, more preferably polyoxypropylene polyols, having a functionality of 1.8 to 3 and an OH number of 20 to 150,
c) at least one chain extender and/or crosslinking agent having a functionality of 2 to 3 and a molecular weight of 62 to 500
in the presence of
d) catalysts,
e) optionally auxiliaries and/or additives,
f) optionally epoxy resins in amounts from 2% to 10% by weight, based on polyurethane.

8. The polyurethane as claimed in claim 7, a tin compound being used as catalyst.

9. The polyurethane as claimed in claim 8, an inorganic tin(IV) compound being used as tin compound.

10. The polyurethane as claimed in claim 7, hollow microspheres being used as additive.

11. Prepolymers containing isocyanate groups and based on diphenylmethane diisocyanate, having an NCO content of 23% to 28% by weight, obtainable from
(i) at least one polyol based on propylene oxide, with an OH number of 200 to 1000, preferably 400 to 800, more preferably 500 to 700 mg KOH/g and a functionality of 1.8 to 4, with the exception of tripropylene glycol, and optionally containing 1,3-butanediol and/or 1,2-propylene glycol in amounts each of max. 2% by weight, based on (a), and
(ii) a mixture of 4,4′-diphenylmethane diisocyanate (4,4′-MDI) modified with carbodiimide groups and uretonimine groups, the uretonimine groups content being 10% to 40% by weight, based on (ii), and a mixture of 15% to 40% by weight of 2,4′-MDI, 60% to 85% by weight of 4,4′-MDI and 0% to 10% by weight of 2,2′-MDI.

12. The use of the polyurethanes as claimed in claim 7 for coating offshore pipes and for producing bend stiffeners, bend restrictors, buoys, clamp systems, cables, flow systems, ballast tanks, and insulation systems.
